(19) United States
(12) Patent Application Publication
(10) Pub. No.: US 2014/0277018 A1
(43) Pub. Date: Sep. 18, 2014

(54) Percutaneous Instrument and Method

(71) Applicant: Ethicon Endo-Surgery, Inc., (US)

(72) Inventor: Shailendra K. Parihar, Mason, OH (US)

(73) Assignee: Ethicon Endo-Surgery, Inc., Cincinnati, OH (US)

(21) Appl. No.: 13/832,496

(22) Filed: Mar. 15, 2013

(51) Int. Cl. A61B 17/295; A61B 17/34

(52) U.S. Cl. CPC A61B 17/295; A61B 17/3478
USPC 606/167

(57) ABSTRACT

A surgical instrument comprises a housing. A pair of tissue manipulating jaws extend distally from the housing. The jaws have an opened position and a closed position. A driver is slideably positioned in the housing and operatively connected to the jaws. The driver has a tissue piercing end projecting proximally from the housing. A percutaneous instrument comprises inner and outer coaxial shafts each having a proximal end and a distal end. An actuator is connected to the proximal ends of the coaxial shafts. Mating features on the distal ends of the coaxial shafts are adapted to engage the driver and housing. A loader comprises an elongate shaft having a proximal end and a distal end. An actuator is attached to the proximal end of the loader elongate shaft. A tube on the distal end of the loader shaft is adapted to releasably hold the end effector.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. The term “endoscopic” refers to all types of minimally invasive surgical procedures including laparoscopic, arthroscopic, natural orifice intraluminal, and natural orifice transluminal procedures. Endoscopic surgery has numerous advantages compared to traditional open surgical procedures, including reduced trauma, faster recovery, reduced risk of infection, and reduced scarring. Endoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars are often used to provide a port through which endoscopic surgical instruments are passed. Trocars generally have an instrument seal, which prevents the insufflatory fluid from escaping while an instrument is positioned in the trocar.

While a variety of different minimally invasive surgical devices are known, no one has previously made or used the surgical devices and methods in accordance with the present invention.

### SUMMARY

In one embodiment, a surgical system comprises an end effector having a tissue manipulating distal end and tissue piercing proximal end. An elongate shaft comprises a proximal end and a distal end adapted for attachment to and detachment from the tissue piercing proximal end of the end effector. An actuator is connected to the proximal end of the elongate shaft.

The surgical system may further comprise a loader sized and adapted to hold the tissue manipulating distal end of the end effector. The loader may comprise an elongate shaft having a proximal end and a distal end, an actuator attached to the proximal end of the loader elongate shaft, and a tube sized to receive the tissue manipulating distal end. The elongate shaft of the loader may articulate.

A surgical method comprises obtaining the end effector; inserting the end effector through a surgical access port and into an insufflated abdominal cavity; while the end effector is in the abdominal cavity, piercing the abdominal wall with the tissue piercing proximal end; obtaining the elongate shaft; and attaching the distal end of the elongate shaft to the tissue piercing proximal end.

In another embodiment, a surgical method comprises obtaining the end effector; inserting the end effector into a body cavity defined by a tissue wall; while the end effector is in the cavity, passing the tissue piercing proximal end through the tissue wall; obtaining the elongate shaft; and attaching the distal end of the elongate shaft to the tissue piercing proximal end while the tissue manipulating distal end is in the cavity. The body cavity may be an abdomen. The body cavity is an thorax. The body cavity is an organ.

In yet another embodiment, a surgical system comprises a first part having a tissue manipulating distal end and tissue piercing proximal end. A second part comprises a shaft having a proximal end and a distal end adapted for attachment to and detachment from the tissue piercing proximal end. An actuator is connected to the proximal end of the elongate shaft. A third part comprises a shaft having a proximal end and a distal end. An actuator is attached to the proximal end of the third part shaft, and a tube is attached to the distal end sized to receive the tissue manipulating distal end.

A surgical method comprises inserting the first part into a body cavity defined by a tissue wall while the tissue manipulating distal end is positioned in the third part tube of claim; manipulating the third part to pass the tissue piercing proximal end through the tissue wall; attaching the distal end of the second part shaft to the tissue piercing proximal end while the tissue manipulating distal end is in the cavity and in the third part tube; and removing the manipulating distal end from the third part tube.

In still another embodiment, a surgical instrument comprises a housing. A pair of tissue manipulating jaws extend distally from the housing. The jaws have an opened position and a closed position. A driver is slideably positioned in the housing and operatively connected to the jaws. The driver has a tissue piercing end projecting proximally from the housing. A percutaneous instrument comprises inner and outer coaxial shafts each having a proximal end and a distal end. An actuator is connected to the proximal ends of the coaxial shafts. Mating features on the distal ends of the coaxial shafts are adapted to engage the driver and housing. A loader comprises an elongate shaft having a proximal end and a distal end. An actuator is attached to the proximal end of the loader elongate shaft. A tube on the distal end of the loader shaft is adapted to releasably hold the end effector.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts a perspective view of an end effector;

FIG. 2 depicts an exploded view on an end effector;

FIG. 3 depicts a perspective view of a loader;

FIG. 4 depicts a perspective view of a percutaneous instrument;

FIG. 5 depicts an insufflated abdomen with an access channel;

FIG. 6 depicts a loader inserted through an access channel;

FIG. 7 depicts an articulated loader inserted through an access channel;

FIG. 8 depicts an end effector piercing the abdominal wall;

FIG. 9 depicts a percutaneous instrument being approximated to the end effector; and

FIG. 10 depicts a percutaneous instrument being attached to an end effector and a loader withdrawn from the abdomen.

### DETAILED DESCRIPTION

FIGS. 1-4 illustrate one example of a surgical system comprising an end effector (100), a loader (200), and a percutaneous instrument (300).

FIG. 1 illustrates one embodiment of an end effector (100). In this example, end effector (100) includes a pair of surgical jaws (110) on the distal end adapted to manipulate tissue. In this embodiment, the jaws (110) are shown as a dissector; however, a variety of other tissue manipulating jaws could also be used, including graspers, sheers, babcocks, forceps, staplers, clip appliers, and the like. Non-jawed end effectors (100) could also be employed such as hook knives, snares, retractors, and the like. In the case of end effectors that require energy, appropriate energy transmission mechanisms known in the art may be added. For instance, appropriate electrical connections may be added between the shaft (320) and end effector (100) to enable bi-polar forceps. Similarly, an ultrasonic transducer and waveguide may be added for the ultrasonic shears end effector.

In the example shown in FIG. 2, the end effector (100) has a housing (130) defined by two shells (132, 134). A pair of jaws (110A, B) pivot about the pin (114) connected to each shell (132, 134). A driver (120) is connected to the jaws (110A, B) by links (112), so axial motion of the driver (120) relative the housing (130) will cause the jaws (110A, B) to move between their open and closed positions. The shells (132, 134) define an opening with a toothed groove (136) adapted to mate to the outer shaft (320). The driver (120) has a proximal end (122) adapted to pierce tissue. In this example proximal end (122) has a sharpened conical point, but other piercing geometries are also contemplated including blades, pyramids, blunted cones, and the like. A groove (124) is adapted to mate to the outer shaft (320).

FIG. 3 illustrates one embodiment of a loader (200). The loader (200) is be used to introduce the end effector (100) into a patient's body cavity, typically through an access port, and to facilitate its attachment and detachment of the end effector (100) to the elongate shaft (320). In this example, the loader (200) has an actuator (210), an elongate shaft (220), and loader tube (240). The loader tube (240) receives the end effector (100) through its distal end (201). A leaf spring (242) releasably holds the end effector (100) in the loader tube (240). In this embodiment the actuator (210) is a longitudinal manual handle.

An input (212) is used to selectively articulate the shaft (220) about the articulation joint (205). A variety of other manual actuators could also be used, including a pistol grip handle, scissor grip handle, a syringe grip handle, endoscopic rotary knobs, and the like. The actuator (210) could also take the form of a robotic interface, such as an DAVINCI puck, a housing comprising gears or pulleys, servomechanisms, and the like. The actuator (210) is operable to articulate the loader tube (240) relative the shaft (220) about the joint (205), as well as to lock and unlock the loader tube (240).

FIG. 4 illustrates one embodiment of a percutaneous instrument (300) comprising an elongate shaft (320) having a distal end (301) and a proximal end (302). The elongate shaft (320) may be rigid and adapted for insertion into a body cavity through an access device, such a trocar, or through direct percutaneous insertion without an access device. The elongate shaft (320) may comprise inner and outer coaxially nested shafts that move relative one another in response to inputs from the actuator (310). The inner and outer shafts (320) each have a mating feature (330) adapted to engage and mate to the grooves (124, 136) in the end effector (100). The mating features (330) can take a variety of forms, including without limitation as leaf spring biasing a mating tooth, threads, collets, interference snap fits, and the like.

The actuator (310) is operably connected to the proximal end (302) of the shaft (320). In this embodiment the actuator (310) is a manual scissor grip handle. The finger grips (312) open and close to axially slide the co-axial shafts relative one another and operate the jaws (110) once the end effector (100) is connected to the shaft (320). The slider (314) extends the outer shaft distally to attached and detach the outer shaft (320) to the end effector (100). The knob (316) rotates the outer shaft (320). A variety of other manual actuators could also be used, including a pistol grip handle, a syringe grip handle, endoscopic rotary knobs, and the like. The actuator (310) could also take the form of a robotic interface, such as an DAVINCI puck, a housing comprising gears or pulleys, servomechanisms, and the like.

FIGS. 5-10 illustrate one example of a sequence of the surgical system being used. As shown in FIG. 5, an abdomen is insufflated and a trocar (500) is inserted through the abdominal wall (400). The end effector (100) is inserted, jaws (110) first, into the loader tube (240). The leaf spring (242) engages the housing (130) to releasable hold the end effector (100) in the loader tube (240). The proximal end (122) of the end effector (100) projects distally from the loader tube (240).

As shown in FIG. 6, the loader tube (240) and end effector (100) are inserted through the trocar (500) and into the abdominal cavity. As shown in FIG. 7, the loader shaft (220) is then articulated. The loader (200) may be rotated and moved until the proximal end (122) of the end effector (100) is approximated to the desired location on the abdominal wall (400). As shown in FIG. 8, the loader (200) is then leveraged to force the proximal end (122) of the end effector (100) to pierce through the abdominal wall (400).

As shown in FIG. 9, the distal end (301) of the percutaneous instrument (300) is approximated to the proximal end (122) of the end effector (100). The end effector (100) is then attached to the shaft (320). The mating feature (330) of the inner shaft (320) mates with the groove (124) on the driver (120) with a snap fit. The slider (314) may be used to distally advance the outer shaft (320) so the corresponding mating feature (330) engages the groove (136) with a snap fit. As shown in FIG. 10, once the end effector (100) is attached to the percutaneous instrument (300), the end effector (100) can be pulled out from the loader tube (240). The loader shaft (220) can then be straightened and removed from the surgical field. A surgeon can then manipulate tissue to perform desired surgical tasks with the jaws (110) by opening and closing the finger grips (312).

The end effector (100) may be disconnected from the percutaneous instrument (300) and removed from the surgical field by reversing the forgoing sequence.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the example above involves an insufflated abdomen, but the teaches herein are equally applicable to any body cavity, including the thorax and pelvis, or any organ. Further, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical system, comprising:
an end effector comprising a tissue manipulating distal end and tissue piercing proximal end;
an elongate shaft comprising a proximal end and a distal end adapted for attachment to and detachment from the tissue piercing proximal end of the end effector; and
an actuator connected to the proximal end of the elongate shaft.

2. The surgical system of claim 1, further comprising a loader sized and adapted to hold the tissue manipulating distal end of the end effector.

3. The surgical system of claim 2, wherein the loader comprises:
an elongate shaft comprising a proximal end and a distal end;
an actuator attached to the proximal end of the loader elongate shaft; and
a tube sized to receive the tissue manipulating distal end.

4. The surgical system of claim 3, wherein the elongate shaft of the loader articulates.

5. A surgical method, comprising:
obtaining the end effector of claim 1;
inserting the end effector through a surgical access port and into an insufflated abdominal cavity;
while the end effector is in the abdominal cavity, piercing the abdominal wall with the tissue piercing proximal end;
obtaining the elongate shaft of claim 1; and
attaching the distal end of the elongate shaft to the tissue piercing proximal end.

6. A surgical method, comprising:
obtaining the end effector of claim 1;
inserting the end effector into a body cavity defined by a tissue wall;
while the end effector is in the cavity, passing the tissue piercing proximal end through the tissue wall;
obtaining the elongate shaft of claim 1; and
attaching the distal end of the elongate shaft to the tissue piercing proximal end while the tissue manipulating distal end is in the cavity.

7. The surgical method of claim 6, wherein the body cavity is an abdomen.

8. The surgical method of claim 6, wherein the body cavity is an thorax.

9. The surgical method of claim 6, wherein the body cavity is an organ.

10. A surgical system, comprising:
a first part comprising a tissue manipulating distal end and tissue piercing proximal end;
a second part comprising a shaft having a proximal end and a distal end adapted for attachment to and detachment from the tissue piercing proximal end, and an actuator connected to the proximal end of the elongate shaft; and
a third part comprising a shaft having a proximal end and a distal end, an actuator attached to the proximal end of the third part shaft, and a tube sized to receive the tissue manipulating distal end.

11. A surgical method, comprising:
inserting the first part of claim 10 into a body cavity defined by a tissue wall while the tissue manipulating distal end is positioned in the third part tube of claim 10;
manipulating the third part to pass the tissue piercing proximal end through the tissue wall;
attaching the distal end of the second part shaft of claim 10 to the tissue piercing proximal end while the tissue manipulating distal end is in the cavity and in the third part tube; and
removing the manipulating distal end from the third part tube.

12. A surgical instrument, comprising:
a housing;
a pair of tissue manipulating jaws extending distally from the housing, the jaws having an opened position and a closed position; and
a driver slideably positioned in the housing and operatively connected to the jaws, the driver comprising a tissue piercing end projecting proximally from the housing.

13. The surgical instrument of claim 12, further comprising a percutaneous instrument comprising inner and outer coaxial shafts each having a proximal end and a distal end, an actuator connected to the proximal ends of the coaxial shafts, and mating features on the distal ends of the coaxial shafts adapted to engage the driver and housing.

14. The surgical instrument of claim 13, further comprising a loader comprising an elongate shaft having a proximal end and a distal end, an actuator attached to the proximal end of the loader elongate shaft, and a tube attached to the distal end of the loader elongate shaft adapted to releasably hold the end effector.
